(19) United States
(12) Patent Application Publication
(10) Pub. No.: US 2022/0240778 A1
(43) Pub. Date: Aug. 4, 2022

(54) Multi-Sensor System For The Intraoral Area

(71) Applicant: Ivoclar Vivadent AG, Schaan (LI)

(72) Inventors: Robert Wolfgang Krybus, Malans (CH); Florian Moosmann, Zwischenwasser (AT)

(21) Appl. No.: 17/588,423

(22) Filed: Jan. 31, 2022

(30) Foreign Application Priority Data
Feb. 3, 2021 (EP) 21155033.0

(51) Int. Cl. A61B 5/00; A61B 5/145; A61B 5/01; A61B 7/00

(52) U.S. Cl. CPC A61B 5/0008; A61B 5/682; A61B 5/6838; A61B 5/7232; A61B 2562/06; A61B 5/14546; A61B 5/14532; A61B 5/01; A61B 7/00; A61B 5/14539

(57) ABSTRACT

A multi-sensor system (100) having a plurality of dental sensors (101-S; 101-M) for arrangement in an oral cavity, including at least one slave dental sensor (101-S) for acquiring measurement data in an oral cavity with a transmitting device (103) for transmitting the measurement data; and a master dental sensor (101-M) with a receiving device (109-M) for receiving the measurement data from the slave dental sensor (101-S).

### CROSS-REFERENCE TO RELATED APPLICATIONS

This application claims priority to European patent application No. 21155033.0 filed on Feb. 3, 2021, the disclosure of which is incorporated herein by reference in its entirety.

### TECHNICAL FIELD

The present invention relates to a multi-sensor system having a plurality of dental sensors for placement in an oral cavity and a method for transmitting measurement data in a multi-sensor system.

### BACKGROUND

The problem with conventional dental sensors is that only one common transmission channel is available to the receiver. As soon as several dental sensors transmit simultaneously, the data may collide. In this case, they cannot be evaluated.

US 20170304024, 20190223751, 20150230885 and 20150064640 are directed to intraoral and/or orthodontic devices and are hereby incorporated by reference in their entirety.

### SUMMARY

It is the technical aim of the present invention to provide a multi-sensor system with several dental sensors, with which measurement data can be transmitted efficiently.

This object is solved by subject-matter according to the independent claims. Technically advantageous embodiments are subject of the dependent claims, the description, and the drawings.

According to a first aspect, the technical problem is solved by a multi-sensor system comprising a plurality of dental sensors for arrangement in an oral cavity, comprising at least one slave dental sensor for acquiring measurement data in the oral cavity with a transmitting device for transmitting the measurement data; and a master dental sensor with a receiving device for receiving the measurement data from the slave dental sensor. The multi-sensor system may include one or more slave dental sensors. The multi-sensor system can achieve efficient, fast, and interference-free data transmission from a plurality of slave dental sensors. The measurement data of all slave dental sensors are received at the master dental sensor and can be bundled and forwarded from this to an external node.

In a technically advantageous embodiment of the multi-sensor system, the slave dental sensor comprises an attachment device or attachment means for attaching the slave dental sensor to a tooth and/or the master dental sensor comprises an attachment device or attachment means for attaching the master dental sensor to a tooth. This has the technical advantage, for example, that the multi-sensor system can be fixed in the mouth of a patient.

In a further technically advantageous embodiment of the multi-sensor system, the transmission device of the slave dental sensor comprises a wired and/or a wireless interface. This has, for example, the technical advantage that the measurement data can be transmitted quickly and easily to the master dental sensor.

In a further technically advantageous embodiment of the multi-sensor system, the slave dental sensor comprises a receiving device for receiving data from the master dental sensor. This has the technical advantage, for example, that additional data can be transmitted from the master dental sensor to the slave dental sensor.

In a further technically advantageous embodiment of the multi-sensor system, the slave dental sensor is designed to be controlled by the master dental sensor. This has the technical advantage, for example, that measurements can be carried out on request by the master dental sensor.

In a further technically advantageous embodiment of the multi-sensor system, the master dental sensor comprises a transmission device for sending the measurement data to a node outside the oral cavity. This has the technical advantage, for example, that the measurement data can be transmitted together on one data channel to the external node for further evaluation and processing.

In a further technically advantageous embodiment of the multi-sensor system, the slave dental sensor and/or the master dental sensor comprise an energy storage device for autonomous operation. This has the technical advantage, for example, that the slave dental sensor or the master dental sensor can be provided without an external energy supply device.

In a further technically advantageous embodiment of the multi-sensor system, the master dental sensor comprises a microprocessor for processing the measurement data. This has the technical advantage, for example, that the measurement data can be processed centrally.

In a further technically advantageous embodiment of the multi-sensor system, the master dental sensor is designed to compress and/or encrypt the measurement data. This has the technical advantage, for example, of reducing the amount of data or carrying out secure communication.

In a further technically advantageous embodiment of the multi-sensor system, the slave dental sensor comprises a digital memory for storing a measurement program and a microprocessor for executing the measurement program. This provides, for example, the technical advantage that the slave dental sensor can be flexibly controlled by a computer program.

In a further technically advantageous embodiment of the multi-sensor system, the master dental sensor is designed to update the measuring program on the slave dental sensor. This has the technical advantage, for example, that better and error-corrected versions of the computer program can be subsequently transmitted to the slave dental sensor.

In a further technically advantageous embodiment of the multi-sensor system, the multi-sensor system is designed to transmit the measurement data to an external application. The application is, for example, a computer program on a smart phone or a tablet PC. The transmission takes place, for example, via a WLAN or a Bluetooth or NFC interface. This has the technical advantage, for example, that the measurement data can be easily visualized on a portable device.

In a further technically advantageous embodiment of the multi-sensor system, the slave dental sensor is designed to detect a pH value, an ethanol concentration, a lactate concentration, a cortisol concentration, a glucose concentration and/or an ion concentration, or to detect sound waves during biting together and/or a temperature. This has the technical advantage, for example, that particularly suitable measurement data from the oral cavity can be acquired by the slave dental sensor.

According to a second aspect, the technical problem is solved by using a multi-sensor system according to the first aspect for acquiring measurement data in an oral cavity. The methods achieve the same technical advantages as the multi-sensor system according to the first aspect.

According to a third aspect, the technical task is solved by a method for transmitting measurement data in a multi-sensor system, comprising the steps of detecting measurement data in an oral cavity by a slave dental sensor; and transmitting the measurement data from the slave dental sensor to a master dental sensor. The method has the same technical advantages as the multi-sensor system according to the first aspect.

In a technically advantageous embodiment of the method, data is sent from the master dental sensor to the slave dental sensor. This also has the technical advantage, for example, that additional data can be transmitted from the master dental sensor to the slave dental sensor.

In a further technically advantageous embodiment of the method, the measurement data is sent from the master dental sensor to a node outside the oral cavity. This also has the technical advantage, for example, that the measurement data can be transmitted together on one data channel to the external node for further evaluation and processing.

### BRIEF DESCRIPTION OF THE DRAWINGS

Examples of embodiments of the invention are shown in the drawings and are described in more detail below.

FIG. 1 shows a schematic view of a multi-sensor system with multiple dental sensors;

FIG. 2 shows another schematic view of the multi-sensor system; and

FIG. 3 shows a block diagram of a method for transmitting measurement data in a multi-sensor system.

### DETAILED DESCRIPTION

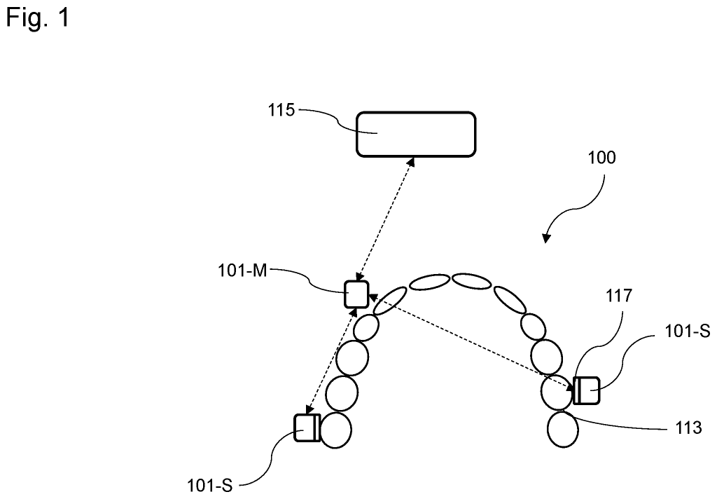

FIG. 1 shows a schematic view of a multi-sensor system 100 with multiple dental sensors 101-S and 101-M. The dental sensors 101-S are slave dental sensors and the dental sensor 101-M is a master dental sensor. The slave dental sensors 101-S and/or the master dental sensor 101-M may be attached to a tooth 113, for example, may be attached using an attachment device or fastener 117 or any attachment means, including, but not limited to an adhesive or cements, and may sense different physical quantities or measurements.

For example, the slave dental sensors 101-S are suitable for detecting a pH value, an ethanol concentration, a lactate concentration, a cortisol concentration, a glucose concentration, and/or an ion concentration as digital measurement data, or for detecting sound waves when biting against each other and/or a temperature as digital measurement data. However, in general, other quantities may be determined by the slave dental sensors 101-S. For this purpose, each of the slave dental sensors 101-S has suitable circuitry and sensor electronics. The electronics of the circuitry acquire and process the measurement data in a suitable manner.

The slave dental sensors 101-S transmit the acquired measurement data to the higher-level master dental sensor 101-M. The master dental sensor 101-M is logically assigned to the slave dental sensors 101-S and is hierarchically superior in data communication. The master dental sensor 101-M masters the access relationships. The multi-sensor system 100 is built around the master dental sensor 101-M. The master dental sensor 101-M receives the measurement data from the respective slave dental sensors 101-S. For this purpose, the master dental sensor 101-M, for example, is the only one of the dental sensors 101 that has the right to access a common communication channel without being requested to do so. In this way, the master dental sensor 101-M can request measurement data from each of the slave dental sensors 101-S in turn. In this way, data collisions that occur when multiple slave dental sensors 101-S attempt to send measurement data simultaneously can be prevented.

The master dental sensor 101-M is in data communication with an external node 115, to which the collected measurement data from the slave dental sensors 101-S is forwarded. At this node 115, processing and display to a user takes place, for example. This node is formed by a computer, for example.

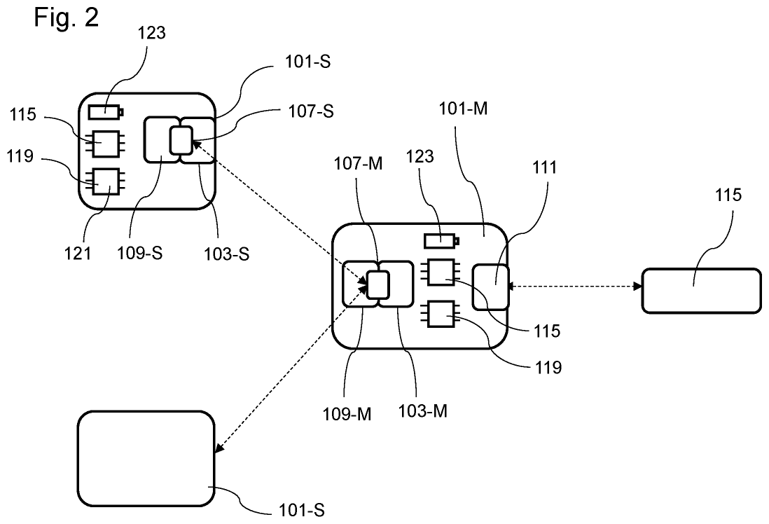

FIG. 2 shows another schematic view of the multi-sensor system 100. The transmitter or transmitting device 103-S of the slave dental sensor 101-S comprises a wired and/or a wireless interface 107-S, which communicates with a corresponding interface 107-M of a receiver or receiving device 109-M of the master dental sensor 101-M. The interfaces 107-S and 107-M communicate with each other, for example, wirelessly via radio, Bluetooth or WLAN protocols or via cable in a bidirectional direction. In this way, measurement data can be transmitted from the slave dental sensor 101-S to the master dental sensor 101-M in one direction. In the other direction, control data or measurement programs can be transmitted from the master dental sensor 101-M to the slave dental sensor 101-S. The interfaces 107-S and interface 107-M can transmit the data bidirectionally, i.e., transmit and receive. A suitable transmission and control protocol can be used for this purpose.

To this end, the slave dental sensor 101-S includes a transmitter/receiver or a transmitting/receiving device 109-S for receiving data from the transmitter/receiver or transmitting/receiving device 103-M of the master dental sensor 101-M. For example, the slave dental sensor 101-S may receive control data from the master dental sensor 101-M via the receiving device 109-S. The control data can, for example, cause the slave dental sensor 101-S to perform a designated measurement and to send the measurement data acquired in the process back to the master dental sensor 101-M. For this purpose, a message is sent from the master dental sensor 101-M to the slave dental sensor 101-S, causing the slave dental sensor 101-S to perform the measurement.

The master dental sensor 101-M includes another transmitting/receiving device 111 for transmitting the measurement data to an external node 115 outside the oral cavity and receiving data. The node 115, which is formed by a PC, for example, can further process the transmitted measurement data and display it to a user.

Both the slave dental sensor 101-S and the master dental sensor 101-M include an electrical energy storage device 123 for autonomous operation. The energy storage 123 supplies electrical energy to the electrical circuits of the slave dental sensor 101-S or the master dental sensor 101-M. The energy storage device 123 is, for example, a battery or an accumulator.

Both the slave dental sensor 101-S and the master dental sensor 101-M comprise a microprocessor 115 for processing the measurement data or for running different computer programs or operating systems. Using this microprocessor 115, the measurement data can be compressed/decompressed and/or decrypted/encrypted. In addition to the microprocessor 115, a digital memory 119 is provided to permanently store the executable measurement programs or the measurement data. For example, the master dental sensor 101-M can update the measurement program 121 on the slave dental sensor 101-S by transferring it from the master dental sensor 101-M to the slave dental sensor 101-S.

Alternatively, the user may determine a sensor to operate as the master dental sensor 101-M by means of the node 115.

Additionally, it is also possible for a multi-master system to be used in which multiple master dental sensors are determined and one or more slave dental sensors are assigned to each master sensor. The multiple master dental sensors can be prioritized by the user to prevent data collision.

Various communication protocols, such as Manchester coding, are suitable for encoding the data. The individual slave or master dental sensors 101-S and 101-M are uniquely addressed by means of the encryption, so that each sensor can be uniquely identified. By means of the node 115, the user can freely select which data is read out, how often and from which sensor. With a suitable application (app), it is possible for the app to evaluate the various measurements, issue warnings or suggest treatment options.

The app can transfer the data to a cloud to which a specific group of people has access, such as dentists or dental technicians. The group of people can access this data and also store data on this cloud, in particular make treatment recommendations, which the user can then view in the app or in the cloud.

Furthermore, the app can process this data for anonymous statistical evaluations. This allows the user to view and compare his teeth with the teeth of other users of the app. The app can also use artificial intelligence to make independent treatment recommendations or detect diseases from the data of different users.

The app can also commission measurements from specific sensors, so that, for example, measurements are first taken in the morning in the area of the molars on one side, at noon in the area of the anterior teeth, and in the evening in the area of the other side of the molars. It is also possible that measurements are only ever taken in the same place for one day.

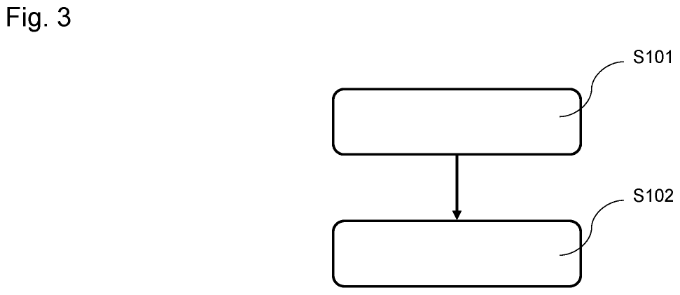

FIG. 3 shows a block diagram of a method for transmitting measurement data in a multi-sensor system 100. In step S101, the measurement data is acquired in the oral cavity by a slave dental sensor 101-S. Then, in step S102, the measurement data is sent from the slave dental sensor 101-S to the master dental sensor 101-M. In this way, the master dental sensor 101-M can collect the measurement data from all the associated slave dental sensors 101-S. For this purpose, the master dental sensor 101-M queries the slave dental sensors 101-S one by one, for example, by means of an addressed message. The master dental sensor 101-M may be configured to control the slave dental sensors 101-S in terms of communication. Then, the master dental sensor 101-M forwards the collected measurement data to the external node 115. The external node 115 may be formed by the app on a smartphone or tablet PC.

All features explained and shown in connection with individual embodiments of the invention may be provided in different combinations in the subject matter of the invention to simultaneously realize their beneficial effects. Any of the elements that form part of the invention are not limited to examples herein and may include those which are understood or known to one of ordinary skill in the art.

All method steps can be implemented by devices which are suitable for executing the respective method step. All functions that are executed by objective features can be a method step of a method.

The scope of protection of the present invention is given by the claims and is not limited by the features explained in the description or shown in the figures.

### REFERENCE LIST

100 Multi-sensor system
101-S Slave dental sensor
101-M Master dental sensor
103-S Transmitter/Transmitting device
103-M Transmitter/Transmitting device
107-S Interface
107-M Interface
109-S Receiver/Receiving device
109-M Receiver/Receiving device
111 Transmitter/Transmitting device
113 Tooth
115 Node
117 Fastener/Fastening device
119 Digital memory
121 Measuring program
123 Energy storage

1. A multi-sensor system (100) having a plurality of dental sensors (101S-; 101-M) for placement in an oral cavity, comprising:
at least one slave dental sensor (101-S) for acquiring measurement data in the oral cavity with a transmitting device (103) for transmitting the measurement data; and
a master dental sensor (101-M) with a receiving device (109-M) for receiving the measurement data from the slave dental sensor (101-S).

2. The multi-sensor system (100) according to claim 1, wherein the slave dental sensor (101-S) comprises a fastener (117) for attaching the slave dental sensor (101-S) to a tooth and/or the master dental sensor (101-M) comprises a fastener (117) for attaching the master dental sensor (101-M) to a tooth.

3. The multi-sensor system (100) according to claim 1, wherein the transmitter (103) of the slave dental sensor (101-S) comprises a wired and/or a wireless interface (107).

4. The multi-sensor system (100) according to claim 1, wherein the slave dental sensor (101-S) comprises a receiver (109) for receiving data from the master dental sensor (101-M).

5. The multi-sensor system (100) according to claim 1, wherein the slave dental sensor (101-S) is adapted to be controlled by the master dental sensor (101-M).

6. The multi-sensor system (100) according to claim 1, wherein the master dental sensor (101-M) comprises a transmitter (111) for transmitting the measurement data to a node (115) outside the oral cavity.

7. The multi-sensor system (100) according to claim 1, wherein the slave dental sensor (101-S) and/or the master dental sensor (101-M) comprise an energy storage for autonomous operation.

8. The multi-sensor system (100) according to claim 1, wherein the master dental sensor (101-M) comprises a microprocessor (115) for processing the measurement data.

9. The multi-sensor system (100) according to claim 1, wherein the master dental sensor (101-M) is configured to compress and/or encrypt the measurement data.

10. The multi-sensor system (100) according to claim 1, wherein the slave dental sensor (101-S) comprises a digital memory (119) for storing a measurement program (121) and a microprocessor (115) for executing the measurement program (121).

11. The multi-sensor system (100) according to claim 1, wherein the multi-sensor system (100) is configured to transmit the measurement data to an external application.

12. The multi-sensor system (100) according to claim 1, wherein the slave dental sensor (101-S) is configured to detect a pH, an ethanol concentration, a lactate concentration, a cortisol concentration, a glucose concentration, and/or an ion concentration, or to detect sound waves during biting together and/or a temperature.

13. A method of using a multi-sensor system (100) comprising at least one slave dental sensor (101-S) for acquiring measurement data in the oral cavity with a transmitting device (103) for transmitting the measurement data and a master dental sensor (101-M) with a receiving device (109-M) for receiving the measurement data from the slave dental sensor (101-S), the method comprising
inserting the multi-sensor system in an oral cavity and acquiring measurement data in the oral cavity.

14. A method for transmitting measurement data in a multi-sensor system (100), comprising the steps:
acquiring (S101) measurement data in an oral cavity by a slave dental sensor (101-S); and
sending (S102) the measurement data from the slave dental sensor (101S-) to a master dental sensor (101-M).

15. The method of claim 14, wherein data is sent from the master dental sensor (101-M) to the slave dental sensor (101-S) and/or the measurement data is sent from the master dental sensor (101-M) to a node (115) outside the oral cavity.
